(19) United States
(12) Patent Application Publication
(10) Pub. No.: US 2015/0374672 A1
(43) Pub. Date: Dec. 31, 2015

(54) PRE-SELECTION OF SUBJECTS FOR THERAPEUTIC TREATMENT WITH AN HSP90 INHIBITORY COMPOUND BASED ON CHEMOSENSITIVE STATUS

(71) Applicant: Synta Pharmaceuticals Corp., Lexington, MA (US)

(72) Inventors: Vojo Vukovic, Winchester, MA (US); Ilker Yalcin, Boston, MA (US)

(21) Appl. No.: 14/707,330

(22) Filed: May 8, 2015

Related U.S. Application Data
(60) Provisional application No. 61/990,454, filed on May 8, 2014.

(51) Int. Cl. A61K 31/4196; A61K 45/06; A61K 31/337

(52) U.S. Cl. CPC A61K 31/4196; A61K 31/337; A61K 45/06

(57) ABSTRACT

The present invention relates to the use of an Hsp90 inhibitor, alone or in combination with another chemotherapeutic agent, in treating cancer in subjects that are determined to be chemosensitive. In particular, the invention features a method of treating cancer in a subject, comprising administering a Hsp90 inhibitor to the subject, wherein the time since diagnosis of cancer in the subject is 6 months or greater.

### RELATED APPLICATIONS

This application claims the benefit of priority to U.S. Provisional Patent Application No. 61/990,454, filed on May 8, 2014, the entire contents of which are hereby incorporated herein by reference.

### FIELD OF THE INVENTION

This invention relates to the use of an Hsp90 inhibitor, alone or in combination with another chemotherapeutic agent, in treating cancer in subjects that are determined to be chemosensitive. Regimens disclosed herein demonstrate potency against certain specific types of cancer, while showing optimal treatment effects in the chemosensitive subject population.

### BACKGROUND OF THE INVENTION

As tumors grow, they begin to exceed their supply of oxygen. Hypoxia occurs when the growth of the tumor exceeds new blood vessel formation, and the tumor must undergo genetic and adaptive changes to allow it to survive and proliferate in a less well-oxygenated environment. In such a hypoxic microenvironment, tumors exhibit a greater dependency on certain signaling pathways, referred to as oxygen-sensitive pathways, to facilitate crucial adaptive mechanisms, such as angiogenesis, glycolysis, growth-factor signaling, immortalization, genetic instability, tissue invasion and metastasis, apoptosis, and pH regulation (see, e.g., Harris, Nature Reviews, 2:38-47, 2002).

Heat shock proteins (HSPs) are a class of chaperone proteins that are up-regulated in response to elevated temperature and other environmental stresses, such as ultraviolet light, nutrient deprivation and oxygen deprivation. HSPs act as chaperones to other cellular proteins (called client proteins), facilitate their proper folding and repair and aid in the refolding of misfolded client proteins. There are several known families of HSPs, each having its own set of client proteins. The Hsp90 family is one of the most abundant HSP families accounting for about 1-2% of proteins in a cell that is not under stress, increasing to about 4-6% in a cell under stress. Inhibition of Hsp90 results in the degradation of its client proteins via the ubiquitin proteasome pathway. Unlike other chaperone proteins, the client proteins of Hsp90 are mostly protein kinases or transcription factors involved in signal transduction, and a number of its client proteins have been shown to be involved in the progression of cancer.

Therapeutic agents targeting these oxygen-sensitive pathways are invaluable for the treatment of diseases such as cancer. However, patient response to currently available therapeutic agents is not always predictable. Indeed, although research has provided physicians with ever more options for therapeutics for the treatment of cancer, the ability to match a therapeutic agent to a specific patient is lacking. Accordingly, a need exists for the accurate prediction of patient response to currently available therapeutic agents

### SUMMARY OF THE INVENTION

It is a novel finding of the present invention that the time since diagnosis of advanced cancer can be used to select a subject population for treatment with an HSP90 inhibitory compound.

Accordingly, in a first aspect the present invention features a method of treating cancer in a subject, comprising administering an effective amount of a Hsp90 inhibitor to the subject, wherein the time since diagnosis of cancer in the subject is 6 months or greater.

In one embodiment, the method comprises the step of selecting a subject in which the time since diagnosis of cancer in the subject is 6 months or greater.

In one embodiment, the cancer is a solid tumor or a hematological malignancy. In a further embodiment, the cancer is advanced cancer.

In one embodiment of the above aspects, the cancer is selected from the group consisting of primary cancer, metastatic cancer, breast cancer, colon cancer, rectal cancer, lung cancer, oropharyngeal cancer, hypopharyngeal cancer, esophageal cancer, stomach cancer, pancreatic cancer, liver cancer, gallbladder cancer, bile duct cancer, small intestine cancer, urinary tract cancer, kidney cancer, bladder cancer, urothelium cancer, female genital tract cancer, cervical cancer, uterine cancer, ovarian cancer, choriocarcinoma, gestational trophoblastic disease, male genital tract cancer, prostate cancer, seminal vesicle cancer, testicular cancer, germ cell tumors, endocrine gland tumors, thyroid cancer, adrenal cancer, pituitary gland cancer, skin cancer, hemangiomas, melanomas, sarcomas arising from bone and soft tissues, Kaposi's sarcoma, brain cancer, nerve cancer, ocular cancer, meningial cancer, astrocytoma, glioma, glioblastoma, retinoblastoma, neuroma, neuroblastoma, Schwannoma, meningioma, solid tumors arising from hematopoietic malignancies, leukemia, Hodgkin's lymphoma, non-Hodgkin's lymphoma, Burkitt's lymphoma, metastatic melanoma, recurrent or persistent ovarian epithelial cancer, fallopian tube cancer, primary peritoneal cancer, epithelial ovarian cancer, primary peritoneal serous cancer, non-small cell lung cancer, gastrointestinal stromal tumors, colorectal cancer, small cell lung cancer (SCLC), melanoma, glioblastoma multiforme, non-squamous non-small-cell lung cancer, malignant glioma, primary peritoneal serous cancer, metastatic liver cancer, neuroendocrine carcinoma, refractory malignancy, triple negative breast cancer, HER2 amplified breast cancer, squamous cell carcinoma, nasopharageal cancer, oral cancer, biliary tract, hepatocellular carcinoma, squamous cell carcinomas of the head and neck (SCCHN), non-medullary thyroid carcinoma, neurofibromatosis type 1, CNS cancer, liposarcoma, leiomyosarcoma, salivary gland cancer, mucosal melanoma, acral/lentiginous melanoma, paraganglioma; pheochromocytoma, advanced metastatic cancer, solid tumor, squamous cell carcinoma, sarcoma, melanoma, endometrial cancer, head and neck cancer, rhabdomysarcoma, multiple myeloma, gastrointestinal stromal tumor, mantle cell lymphoma, gliosarcoma, bone sarcoma, and refractory malignancy. In a particular embodiment, the cancer is non-small cell lung carcinoma (NSCLC).

In one embodiment of the above aspects, the Hsp90 inhibitor is administered in combination with one or more chemotherapeutic agents. In a related embodiment, the chemotherapeutic agent is administered at the same time as Hsp90. In a further embodiment, the chemotherapeutic agent is administered after Hsp90. In another further embodiment, the Hsp90 inhibitor is Genetespib. In another embodiment, the chemotherapeutic agent is selected from the group consisting of the taxanes (paclitaxel and docetaxel), fulvestrant, crizotinib, adriamycin (doxorubicin), cisplatin, camptothecin, 5-fluorouracil, analogs thereof, and other chemotherapeutic agents which demonstrate activity against tumours ex vivo and in vivo. In a preferred embodiment, the chemotherapeutic agent is docetaxel.

In one embodiment of the above aspects, the amount of the Hsp90 inhibitor administered is from 75 mg/m² to 260 mg/m². In a further embodiment, the amount of the Hsp90 inhibitor administered is from 125 mg/m² to 260 mg/m². In another further embodiment, the amount of the Hsp90 inhibitor administered is from 175 mg/m² to 260 mg/m². In still another further embodiment, the amount of the Hsp90 inhibitor administered is 75 mg/m², 85 mg/m², 100 mg/m², 110 mg/m², 115 mg/m², 120 mg/m², 145 mg/m², 150 mg/m², 175 mg/m², 180 mg/m², 200 mg/m², 215 mg/m² or 260 mg/m². In one embodiment of the above aspects, the Hsp90 inhibitor is administered by intravenous infusion. In a further embodiment, the infusion is a peripheral intravenous infusion. In another further embodiment, the Hsp90 inhibitor is infused over 60 minutes.

In another aspect, the invention features a method of treating advanced NSCLC in a subject, comprising selecting a subject in which the time since diagnosis of cancer in the subject is 6 months or greater; and administering Genetespib to the subject in combination with docetaxel, thereby treating advanced NSCLC in the subject.

The invention also features a kit to practice the methods of any one of the embodiments herein.

### BRIEF DESCRIPTION OF THE DRAWINGS

FIG. 1 shows an Overall Survival (OS) Kaplan Meier plot for the chemosensitive patient population of GALAXY-1 selected for evaluation in the GALAXY-2 Phase 3 trial.

FIG. 2 shows a Progression-Free Survival (PFS) Kaplan Meier plot for the chemosensitive patient population of GALAXY-1 selected for evaluation in the GALAXY-2 Phase 3 trial.

FIG. 3 shows a Table that summarizes the effect of prognostic factors on Overall Survival (OS) in the adenocarcinoma analysis population (Randomized Patients) [N=253].

FIG. 4 shows a Table that summarizes the effect of prognostic factors on Overall Survival (OS) in adenocarcinoma patients with diagnosis of advanced disease>6 months (Randomized Patients) [N=177].

FIG. 5 is a graph that shows various cut-offs (by month) and corresponding hazard ratio (HR) estimates.

FIG. 6 is a graph that shows an overall survival (OS) forest plot for the chemosensitive population.

### DETAILED DESCRIPTION OF THE INVENTION

Research has provided the physician with ever more options for therapeutics for the treatment of cancer. However, despite the availability of the new agents, the ability to match a therapeutic agent to a specific patient based not just on the type of tumor or site of the tumor, but the characteristic of the tumor, is lacking.

The instant invention provides methods of identifying a subject who will likely respond favorably to treatment with an Hsp90 inhibitor by determining the level of hypoxia in a tumor, either by looking directly at markers within the tumor tissue or looking at markers in a peripheral sample from the subject, e.g., a bodily fluid such as blood, serum, plasma, lymph, urine, cerebrospinal fluid, fecal matter, circulating tumor cells, bronchial lavage, peritoneal lavage, exudate, effusion, and sputum for the presence of one or more indicators of the level of hypoxia in the tumor.

Hsp90 inhibitors affect hypoxia-driven pathways, including VEGF and mTOR. For example, Hsp90 inhibitors inhibit HIF1. Further, several key elements of the VEGF and mTOR pathways are client proteins VEGF, VEGFR1-3, IGF-1R, GLUT1-3, PI3K of Hsp90. Ganetespib has been shown to down-regulate the expression or phosphorylation of Hsp90 client proteins. Therefore, Hsp90 inhibitors are be useful in the treatment of subjects with cancer wherein the tumor has a high level of hypoxia.

The present invention provides the use of an Hsp90 inhibitor, alone or in combination with another chemotherapeutic agent, in treating cancer in subjects that are determined to be chemosensitive. In particular, the present invention provides a method of treating cancer in a subject, comprising administering a Hsp90 inhibitor to the subject, wherein the time since diagnosis of cancer in the subject is 6 months or greater.

### Definitions

Unless otherwise specified, the below terms used herein are defined as follows:

The articles “a”, “an” and “the” are used herein to refer to one or to more than one (i.e., to at least one) of the grammatical object of the article unless otherwise clearly indicated by contrast. By way of example, “an element” means one element or more than one element.

The term “including” is used herein to mean, and is used interchangeably with, the phrase “including but not limited to”.

The term “or” is used herein to mean, and is used interchangeably with, the term “and/or,” unless context clearly indicates otherwise.

The term “such as” is used herein to mean, and is used interchangeably, with the phrase “such as but not limited to”.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

The recitation of a listing of chemical group(s) in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

As used herein, the term “subject” refers to human and non-human animals, including veterinary subjects. The term “non-human animal” includes all vertebrates, e.g., mammals and non-mammals, such as non-human primates, mice, rabbits, sheep, dog, cat, horse, cow, chickens, amphibians, and reptiles. In a preferred embodiment, the subject is a human and may be referred to as a patient.

As used herein, the terms “treat,” “treating” or “treatment” refer, preferably, to an action to obtain a beneficial or desired clinical result including, but not limited to, alleviation or amelioration of one or more signs or symptoms of a disease or condition, diminishing the extent of disease, stability (i.e., not worsening) state of disease, amelioration or palliation of the disease state, diminishing rate of or time to progression, and remission (whether partial or total), whether detectable or undetectable.

The terms “treat”, “treatment” and “treating” include the reduction or amelioration of the progression, severity and/or duration of cancer, or the amelioration of one or more symptoms of cancer, resulting from the administration of the compound of formula (I). The terms “treat”, “treatment” and “treating” also include the reduction of the risk of recurrence of cancer or the delay or inhibition of the recurrence of cancer. In an embodiment, the terms “treat”, “treatment” and “treating” include the amelioration of at least one measurable physical parameter of cancer, such as growth of a tumor, not necessarily discernible by the patient. In other embodiments, the terms “treat”, “treatment” and “treating” includes the inhibition of the progression of cancer either physically by the stabilization of a discernible symptom, physiologically by the stabilization of a physical parameter, or both. In another embodiment, the terms “treat”, “treatment” and “treating” of cancer include the reduction or stabilization of tumor size or cancerous cell count, and/or delay of tumor formation. “Treatment” can also mean prolonging survival as compared to expected survival in the absence of treatment. Treatment does not need to be curative.

The term “chemosensitive,” “chemosensitive subject” or “chemosensitive subject population,” as used herein, refers to subjects in which the time since diagnosis of cancer in the subject is 6 months or greater (that is, at least 6 months have elapsed since diagnosis of advanced disease). In exemplary embodiments, a chemosensitive subject is one in which the time since diagnosis of advanced non-small cell lung cancer (NSCLC) is 6 months or greater. In particular embodiments, a chemosensitive subject or subject population has been previously treated with a chemotherapeutic agent (i.e. has received first line treatment).

By “diagnosing” and the like, as used herein, refers to a clinical or other assessment of the condition of a subject based on observation, testing, or circumstances for identifying a subject having a disease, disorder, or condition based on the presence of at least one indicator, such as a sign or symptom of the disease, disorder, or condition.

Typically, diagnosing using the method of the invention includes the observation of the subject for multiple indicators of the disease, disorder, or condition in conjunction with the methods provided herein. Diagnostic methods provide an indicator that a disease is or is not present. A single diagnostic test typically does not provide a definitive conclusion regarding the disease state of the subject being tested.

In embodiments of the present invention, the time since diagnosis of a subject with advanced cancer is 6 months or greater.

The terms “administer”, “administering” or “administration” include any method of delivery of a pharmaceutical composition or agent into a subject's system or to a particular region in or on a subject. In certain embodiments of the invention, an agent is administered intravenously, intramuscularly, subcutaneously, intradermally, intranasally, orally, transcutaneously, or mucosally. In a preferred embodiment, an agent is administered intravenously. Administering an agent can be performed by a number of people working in concert. Administering an agent includes, for example, prescribing an agent to be administered to a subject and/or providing instructions, directly or through another, to take a specific agent, either by self-delivery, e.g., as by oral delivery, subcutaneous delivery, intravenous delivery through a central line, etc.; or for delivery by a trained professional, e.g., intravenous delivery, intramuscular delivery, intratumoral delivery, etc.

As used herein, the term “survival” refers to the continuation of life of a subject which has been treated for a disease or condition, e.g., cancer.

As used herein, the term “recur” refers to the re-growth of tumor or cancerous cells in a subject in whom primary treatment for the tumor has been administered. The tumor may recur in the original site or in another part of the body. In one embodiment, a tumor that recurs is of the same type as the original tumor for which the subject was treated. For example, if a subject had an ovarian cancer tumor, was treated and subsequently developed another ovarian cancer tumor, the tumor has recurred. In addition, a cancer can recur in or metastasize to a different organ or tissue than the one where it originally occurred.

As used herein, the terms “identify” or “select” refer to a choice in preference to another. In other words, to identify a subject or select a subject is to perform the active step of picking out that particular subject from a group and confirming the identity of the subject by name or other distinguishing feature. With respect to the instant invention, it is understood that selecting a subject in which the time since diagnosis of cancer in the subject is 6 months or greater can include any of a number of acts including, but not limited to, performing a test and observing a result that is indicative of a subject having cancer for 6 months or greater; reviewing a test result of a subject and identifying the subject as having cancer for 6 months or greater; reviewing documentation, such as medical records, on a subject stating that the subject has had cancer for 6 months or greater.

As used herein, the term “benefit” refers to something that is advantageous or good, or an advantage. Similarly, the term “benefiting”, as used herein, refers to something that improves or advantages. For example, a subject will benefit from treatment if the time since diagnosis of cancer in the subject is 6 months or greater. A benefit can also include an improvement in quality of life, or an increase in survival time or progression free survival.

The terms “cancer” or “tumor” are well known in the art and refer to the presence, e.g., in a subject, of cells possessing characteristics typical of cancer-causing cells, such as uncontrolled proliferation, immortality, metastatic potential, rapid growth and proliferation rate, decreased cell death/apoptosis, and certain characteristic morphological features. Cancer cells are often in the form of a solid tumor. However, cancer also includes non-solid tumors, e.g., blood tumors, e.g., leukemia, wherein the cancer cells are derived from bone marrow. As used herein, the term “cancer” includes pre-malignant as well as malignant cancers. Cancers include, but are not limited to, acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute T-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myelogenous leukemia, colon cancer, colorectal cancer, craniopharyngioma, cystadenocarcinoma, diffuse large B-cell lymphoma, Burkitt's lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, heavy chain disease, hemangioblastoma, hepatoma, hepatocellular cancer, hormone insensitive prostate cancer, leiomyosarcoma, liposarcoma, lung cancer, lymphagioendotheliosarcoma, lymphangiosarcoma, lymphoblastic leukemia, lymphoma (Hodgkin's and non-Hodgkin's), malignancies and hyperproliferative disorders of the bladder, breast, colon, lung, ovaries, pancreas, prostate, skin, and uterus, lymphoid malignancies of T-cell or B-cell origin, leukemia, lymphoma, medullary carcinoma, medulloblastoma, melanoma, meningioma, mesothelioma, multiple myeloma, myelogenous leukemia, myeloma, myxosarcoma, neuroblastoma, non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer, and Wilms' tumor. Other cancers include primary cancer, metastatic cancer, oropharyngeal cancer, hypopharyngeal cancer, liver cancer, gall bladder cancer, bile duct cancer, small intestine cancer, urinary tract cancer, kidney cancer, urothelium cancer, female genital tract cancer, uterine cancer, gestational trophoblastic disease, male genital tract cancer, seminal vesicle cancer, testicular cancer, germ cell tumors, endocrine gland tumors, thyroid cancer, adrenal cancer, pituitary gland cancer, hemangioma, sarcoma arising from bone and soft tissues, Kaposi's sarcoma, nerve cancer, ocular cancer, meningial cancer, glioblastomas, neuromas, neuroblastomas, Schwannomas, solid tumors arising from hematopoietic malignancies such as leukemias, metastatic melanoma, recurrent or persistent ovarian epithelial cancer, fallopian tube cancer, primary peritoneal cancer, gastrointestinal stromal tumors, colorectal cancer, gastric cancer, melanoma, glioblastoma multiforme, non-squamous non-small-cell lung cancer, malignant glioma, epithelial ovarian cancer, primary peritoneal serous cancer, metastatic liver cancer, neuroendocrine carcinoma, refractory malignancy, triple negative breast cancer, HER2 amplified breast cancer, nasopharageal cancer, oral cancer, biliary tract, hepatocellular carcinoma, squamous cell carcinomas of the head and neck (SCCHN), non-medullary thyroid carcinoma, recurrent glioblastoma multiforme, neurofibromatosis type 1, CNS cancer, liposarcoma, leiomyosarcoma, salivary gland cancer, mucosal melanoma, acral/lentiginous melanoma, paraganglioma, pheochromocytoma, advanced metastatic cancer, solid tumor, triple negative breast cancer, colorectal cancer, sarcoma, melanoma, renal carcinoma, endometrial cancer, thyroid cancer, rhabdomysarcoma, multiple myeloma, ovarian cancer, glioblastoma, gastrointestinal stromal tumor, mantle cell lymphoma, and refractory malignancy. In particular embodiments, the cancer is NSCLC.

“Solid tumor,” as used herein, is understood as any pathogenic tumor that can be palpated or detected using imaging methods as an abnormal growth having three dimensions. A solid tumor is differentiated from a blood tumor such as leukemia. However, cells of a blood tumor are derived from bone marrow; therefore, the tissue producing the cancer cells is a solid tissue that can be hypoxic.

“Tumor tissue” is understood as cells, extracellular matrix, and other naturally occurring components associated with the solid tumor.

As used herein, the terms “hypoxia” and “hypoxic” refer to a condition in which a cancer or a tumor has a low oxygen microenvironment or a less well-oxygenated microenvironment. Hypoxia occurs when tumor growth exceeds new blood vessel formation, and a tumor must undergo genetic and adaptive changes to allow them to survive and proliferate in the hypoxic environment. The development of intratumoral hypoxia is a common sign of solid tumors. When a tumor microenvironment is less well-oxygenated, there is a greater dependency on oxygen-sensitive pathways, including but not limited to HIF1 pathways, VEGF pathways, and mTOR pathways. These pathways facilitate crucial adaptive mechanisms, such as angiogenesis, glycolysis, growth-factor signaling, immortalization, genetic instability, tissue invasion and metastasis, apoptosis, and pH regulation (see, e.g., Harris, Nature Reviews, 2:38-47, 2002). These pathways may also facilitate invasion and metastasis. Accordingly, the treatment of a subject with a cancer or tumor with a selected agent such as bevacizumab, ganetespib, temsirolimus, erlotinib, PTK787, BEZ235, XL765, pazopanib, cediranib, or axitinib is more effective when the subject has a tumor that exhibits a modulated level of hypoxia, e.g., a high level of hypoxia. As the level of hypoxia in the tumor can be determined by obtaining a sample from a site other than the tumor, as used herein, the subject can be stated to demonstrate a modulated level of hypoxia when it is the tumor present in the subject that demonstrates a modulated level of hypoxia. As used herein it is understood that the subject with a modulated level of hypoxia is typically not suffering from systemic oxygen imbalance or ischemic disease at a site remote from the tumor.

As used herein, the term “control sample,” as used herein, refers to any clinically relevant comparative sample, including, for example, a sample from a healthy subject not afflicted with cancer, a sample from a subject having a less severe or slower progressing cancer than the subject to be assessed, a sample from a subject having some other type of cancer or disease, a sample from a subject prior to treatment, a sample of non-diseased tissue (e.g., non-tumor tissue), a sample from the same origin and close to the tumor site, and the like. A control sample can be a purified sample, protein, and/or nucleic acid provided with a kit. Such control samples can be diluted, for example, in a dilution series to allow for quantitative measurement of analytes in test samples. A control sample may include a sample derived from one or more subjects. A control sample may also be a sample made at an earlier time point from the subject to be assessed. For example, the control sample could be a sample taken from the subject to be assessed before the onset of the cancer, at an earlier stage of disease, or before the administration of treatment or of a portion of treatment. The control sample may also be a sample from an animal model, or from a tissue or cell lines derived from the animal model, of the cancer. The level of signal detected or protein expression in a control sample that consists of a group of measurements may be determined, e.g., based on any appropriate statistical measure, such as, for example, measures of central tendency including average, median, or modal values.

As used herein, “changed as compared to a control” sample or subject is understood as having a level of the analyte or diagnostic or therapeutic indicator to be detected at a level that is statistically different than a sample from a normal, untreated, or control sample. As used herein, “determining” is understood as performing an assay or using a diagnostic method to ascertain the state of someone or something, e.g., the presence, absence, level, or degree of a certain condition, biomarker, disease state, or physiological condition.

“Prescribing” as used herein is understood as indicating a specific agent or agents for administration to a subject.

As used herein, the terms “respond” or “response” are understood as having a positive response to treatment with a therapeutic agent, wherein a positive response is understood as having a decrease in at least one sign or symptom of a disease or condition (e.g., tumor shrinkage, decrease in tumor burden, inhibition or decrease of metastasis, improving quality of life (“QOL”), delay of time to progression (“TTP”), increase of overall survival (“OS”), etc.), or slowing or stopping of disease progression (e.g., halting tumor growth or metastasis, or slowing the rate of tumor growth or metastasis). A response can also include an improvement in quality of life, or an increase in survival time or progression free survival.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Reference will now be made in detail to preferred embodiments of the invention. While the invention will be described in conjunction with the preferred embodiments, it will be understood that it is not intended to limit the invention to those preferred embodiments. To the contrary, it is intended to cover alternatives, modifications, and equivalents as may be included within the spirit and scope of the invention as defined by the appended claims.

Methods of Treatment

The present invention features methods of treating cancer in a subject, comprising administering a Hsp90 inhibitor to the subject, wherein the time since diagnosis of cancer in the subject is 6 months or greater. In particular embodiment, the methods comprise the step of selecting a subject in which the time since diagnosis of cancer in the subject is 6 months or greater.

In particular, the present invention is based on the novel and surprising finding that advanced cancer can be treated in a subject in which the time since diagnosis of cancer in the subject is 6 months or greater. Accordingly, the invention features the step of selecting a subject in which the time since diagnosis of cancer in the subject is 6 months or greater.

A “chemosensitive subject” is meant to refer to subjects in which the time since diagnosis of cancer in the subject is 6 months or greater (that is, at least 6 months have elapsed since diagnosis of advanced disease).

Cancers that may be treated or prevented using the methods of the invention include, for example, acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute T-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myleogeneous leukemia, colon cancer, colorectal cancer, craniopharyngioma, cystadenocarcinoma, diffuse large B-cell lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, heavy chain disease, hemangioblastoma, hepatoma, hepatocellular cancer, hormone insensitive prostate cancer, leiomyosarcoma, liposarcoma, lung cancer, lymphagioendotheliosarcoma, lymphangiosarcoma, lymphoblastic leukemia, lymphoma (Hodgkin's and non-Hodgkin's), malignancies and hyperproliferative disorders of the bladder, breast, colon, lung, ovaries, pancreas, prostate, skin and uterus, lymphoid malignancies of T-cell or B-cell origin, leukemia, lymphoma, medullary carcinoma, medulloblastoma, melanoma, meningioma, mesothelioma, multiple myeloma, myelogenous leukemia, myeloma, myxosarcoma, neuroblastoma, non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer and Wilms' tumor. Other cancers include primary cancer, metastatic cancer, oropharyngeal cancer, hypopharyngeal cancer, liver cancer, gallbladder cancer, small intestine cancer, urinary tract cancer, kidney cancer, urothelium cancer, female genital tract cancer, uterine cancer, gestational trophoblastic disease, male genital tract cancer, seminal vesicle cancer, testicular cancer, germ cell tumors, endocrine gland tumors, thyroid cancer, adrenal cancer, and pituitary gland cancer, hemangiomas, sarcomas arising from bone and soft tissues; Kaposi's sarcoma, nerve cancer, ocular cancer, and meningial cancer, glioblastomas, neuromas, Schwannomas, solid tumors arising from hematopoietic malignancies such as leukemias, metastatic melanoma, recurrent or persistent ovarian epithelial cancer, fallopian tube cancer, primary peritoneal cancer, gastrointestinal stromal tumors, colorectal cancer, gastric cancer, melanoma, glioblastoma multiforme, non-squamous non-small-cell lung cancer, malignant glioma, epithelial ovarian cancer, primary peritoneal serous cancer, metastatic liver cancer, neuroendocrine carcinoma, refractory malignancy, triple negative breast cancer, HER2 amplified breast cancer, squamous cell carcinoma of the head and neck (SCCHN), nasopharageal cancer, oral cancer, biliary tract, hepatocellular carcinoma, non-medullary thyroid carcinoma, recurrent glioblastoma multiforme, neurofibromatosis type 1, CNS cancer, liposarcoma; leiomyosarcoma; salivary gland cancer, mucosal melanoma; acral/lentiginous melanoma, paraganglioma, and pheochromocytoma.

In preferred embodiments, the cancer is non-small cell lung carcinoma (NSCLC).

It is understood that diagnosis and treatment of a complex disease such as cancer is not performed by a single individual, test, agent, or intervention. For example, a subject may meet with a primary care physician to express a concern and be referred to an oncologist who will request tests that are designed, carried out, and analyzed by any of a number of individuals, but not limited to, radiologists, radiology technicians, physicists, phlebotomists, pathologists, laboratory technicians, and radiation, clinical, and surgical oncologists. Selection, dosing, and administration of agents to a subject diagnosed with cancer will be performed by any of a number of individuals including, but not limited to, radiologists, radiology technicians, physicists, pathologists, infusion nurses, pharmacists, and radiation, clinical, and surgical oncologists. Similarly, administering an agent can be performed by a number of people working in concert. Administering an agent includes, for example, prescribing an agent to be administered to a subject and/or providing instructions, directly or through another, to take a specific agent, either by self-delivery, e.g., as by oral delivery, subcutaneous delivery, intravenous delivery through a central line, etc.; or for delivery by a trained professional, e.g., intravenous delivery, intramuscular delivery, intratumoral delivery, etc.

Hsp90 Inhibitors

As used herein, an “Hsp90 inhibitor” is understood as a therapeutic agent that reduces the activity of Hsp90 either by directly interacting with Hsp90 or by preventing the formation of the Hsp90/CDC37 complex such that the expression and proper folding of at least one client protein of Hsp90 is inhibited. “Hsp90” includes each member of the family of heat shock proteins having a mass of about 90-kilodaltons. For example, in humans the highly conserved Hsp90 family includes cytosolic Hsp90 and Hsp90 isoforms, as well as GRP94, which is found in the endoplasmic reticulum, and HSP75/TRAP1, which is found in the mitochondrial matrix. As used herein, Hsp90 inhibitors include, but are not limited to ganetespib, geldanamycin (tanespimycin), e.g., IPI-493, macbecins, tripterins, tanespimycins, e.g., 17-AAG (alvespimycin), KF-55823, radicicols, KF-58333, KF-58332, 17-DMAG, IPI-504, BI1B-021, BI1B-028, PU-H64, PU-H71, PU-DZ8, PU-HZ151, SNX-2112, SNX-2321, SNX-5422, SNX-7081, SNX-8891, SNX-0723, SAR-567530, ABI-287, ABI-328, AT-13387, NSC-113497, PF-3823863, PF-4470296, EC-102, EC-154, ARQ-250-RP, BC-274, VER-50589, KW-2478, BHI-001, AUY-922, EMD-614684, EMD-683671, XL-888, VER-51047, KOS-2484, KOS-2539, CUDC-305, MPC-3100, CH-5164840, PU-DZ13, PU-HZ151, PU-25 DZ13, VER-82576, VER-82160, VER-82576, VER-82160, NXD-30001, NVP-HSP990, SST-0201CL1, SST-0115AA1, SST-0221AA1, SST-0223AA1, novobiocin, herbinmycin A, radicicol, CCT018059, PU-H71, and celastrol. In certain embodiments, Hsp90 inhibitors do not include ganetespib.

In certain embodiments, the Hsp90 inhibitor is a compound of formula (I) or a pharmaceutically acceptable salt or tautomer thereof:

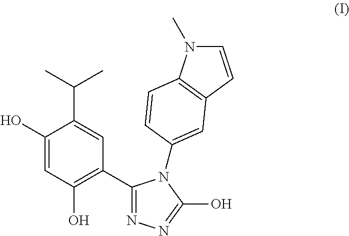 [SMILES: CC(C)c1cc(-c2nnc(O)n2-c2ccc3c(ccn3C)c2)c(O)cc1O]

In certain embodiments, the Hsp90 inhibitor is Ganetespib. Ganetespib is a resorcinolic triazolone compound that competitively binds to the ATP pocket of the N-terminus of heat shock protein 90 (Hsp90), thus blocking Hsp90 chaperone activity [Ying, Mol Cancer Ther, 2012]. Ganetespib is structurally distinct from the first-generation ansamycin Hsp90 inhibitors. With a molecular weight of 364.4, ganetespib is considerably smaller than the ansamycin class and most of the newer, second-generation Hsp90 inhibitors. Ganetespib is relatively hydrophobic, with a c Log P value of 3.3. The X-ray co-crystal structure of ganetespib bound to Hsp90 confirmed important hydrogen bonding interactions, also seen in the ansamycin family, involving the resorcinol hydroxyl group with Asp93 and the carbonyl group of triazolone with Lys58. Importantly, in ganetespib, the 2-hydroxyl of resorcinol is within hydrogen bonding distance to both oxygen atoms of the carboxylic group in Asp93, resulting in a substantially stronger interaction. Furthermore, the N2 of triazolone forms a water-bridged hydrogen bond with Asp93 to provide additional hydrogen bonding. Water-bridge hydrogen bonds between 4-hydroxyl of resorcinol and Leu48 and Ser52 are critical for binding efficiency. The hydrazine carboxamide moiety of triazolone in ganetespib is of particular structural importance. In addition to the direct hydrogen bond with Lys58, it forms a unique hydrogen bond with Gly97, a distinguishing feature from the ansamycin analogues. Furthermore, it interacts with Thr184 and Asp102 through water-bridge hydrogen bonding.

Exemplary Hsp90 inhibitors include those disclosed in U.S. Pat. Nos. 8,362,055 and 7,825,148, and WO2012155063 A1, each of which is incorporated by reference in its entirety herein.

In certain embodiments, the Hsp90 inhibitor is administered with one or more additional chemotherapeutic agents.

A “chemotherapeutic agent” is understood as any drug used for the treatment of cancer. Chemotherapeutic agents include, but are not limited to, small molecules and biologics (e.g., antibodies, peptide drugs, nucleic acid drugs).

Such chemotherapeutic agents include the taxanes, fulvestrant, crizotinib, adriamycin (also known as doxorubicin), cisplatin, paclitaxel, camptothecin, 5-fluorouracil, analogs thereof, and other chemotherapeutic agents which demonstrate activity against tumours ex vivo and in vivo. Such chemotherapeutic agents also include alkylating agents, antimetabolites, natural products (such as vinca alkaloids, epidophyllotoxins, antibiotics, enzymes and biological response modifiers), topoisomerase inhibitors, microtubule inhibitors, spindle poisons, hormones and antagonists, and miscellaneous agents such as platinum coordination complexes, anthracendiones, substituted ureas, etc. those of skill in the art will know of other chemotherapeutic agents.

The taxanes are anti-cancer agents that include paclitaxel (TAXOL) and docetaxel (TAXOTERE). Both drugs have proved to be effective in the treatment of a variety of solid tumors including breast, ovarian, lung, and bladder cancers. Thus, the term “paclitaxel analog” is defined herein to mean a compound which has the basic paclitaxel skeleton and which stabilizes microtubule formation. Many analogs of paclitaxel are known, including docetaxel. In addition, a paclitaxel analog can also be bonded to or be pendent from a pharmaceutically acceptable polymer, such as a poly aery lamide. The term “paclitaxel analog”, as it is used herein, includes such polymer linked taxanes.

In particular preferred embodiments, the chemotherapeutic agent is docetaxel.

Accordingly, in exemplary embodiments the invention features a method of treating advanced NSCLC in a subject, comprising selecting a subject in which the time since diagnosis of cancer in the subject is 6 months or greater; and administering Genetespib to the subject in combination with docetaxel, thereby treating advanced NSCLC in the subject.

The dosages of other anti-cancer agents, which have been or are currently being used to prevent, treat, manage, or ameliorate disorders, such cancer, or one or more symptoms thereof can be used in the combination therapies of the invention. Preferably, dosages lower than those which have been or are currently being used to prevent, treat, manage, or ameliorate cancer, or one or more symptoms thereof, are used in the combination therapies of the invention. The recommended dosages of agents currently used for the prevention, treatment, management, or amelioration of cancer, or one or more symptoms thereof, can obtained from any reference in the art including, but not limited to, Hardman et al., eds., 1996, Goodman & Gilman's The Pharmacological Basis Of Basis Of Therapeutics 9th Ed, Mc-Graw-Hill, New York; Physician's Desk Reference (PDR) 57^{th} Ed., 2003, Medical Economics Co., Inc., Montvale, N.J.

An “effective amount” is that amount sufficient to treat a disease in a subject. A therapeutically effective amount can be administered in one or more administrations.

The term “effective amount” includes an amount of the compound of formula (I) which is sufficient to treat the cancer, to reduce or ameliorate the severity, duration, or progression of cancer, to retard or halt the advancement of cancer, to cause the regression of cancer, to delay the recurrence, development, onset, or progression of a symptom associated with cancer, or to enhance or improve the therapeutic effect(s) of another therapy. For example, an effective amount can induce, for example, a complete response, a partial response, or stable disease; as determined, for example, using RESIST criteria.

An “effective amount” of a therapeutic agent produces a desired response. Having a positive response to treatment with a therapeutic agent is understood as having a decrease in at least one sign or symptom of a disease or condition {e.g., tumor shrinkage, decrease in tumor burden, inhibition or decrease of metastasis, improving quality of life (“QOL”), delay of time to progression (“TTP”), increase of overall survival (“OS”), etc.), or slowing or stopping of disease progression (e.g., halting tumor growth or metastasis, or slowing the rate of tumor growth or metastasis). It is understood that an “effective amount” need not be curative.

An effective amount of an Hsp90 inhibitor is understood as an amount of an Hsp90 inhibitor to improve outcome relative to an appropriate control group, e.g., an untreated group, a group treated with a combination of therapies not including the an Hsp90 inhibitor. Methods to select appropriate control groups and to perform comparative analyses are within the ability of those of skill in the art.

The precise amount of compound administered to provide an “effective amount” of an Hsp90 inhibitor to the subject will depend on the mode of administration, the type and severity of the cancer and on the characteristics of the subject, such as general health, age, sex, body weight and tolerance to drugs. The skilled artisan will be able to determine appropriate dosages depending on these and other factors. When administered in combination with other therapeutic agents, e.g., when administered in combination with an anti-cancer agent, an “effective amount” of any additional therapeutic agent(s) will depend on the type of drug used. Suitable dosages are known for approved therapeutic agents and can be adjusted by the skilled artisan according to the condition of the subject, the type of condition(s) being treated and the amount of a compound of the invention being used by following, for example, dosages reported in the literature and recommended in the Physician's Desk Reference (57th ed., 2003).

In certain embodiments, the Hsp90 inhibitor is Ganetespib, and the amount of Ganetespib administered will depend upon the patient's body surface area (BSA). In other preferred embodiments, Gantespib is administered intravenously.

The dosage of an individual agent used in combination therapy may be equal to or lower than the dose of an individual therapeutic agent when given independently to treat, manage, or ameliorate a disease or disorder, or one or more symptoms thereof. In one embodiment, the disease or disorder being treated with a combination therapy is a triple-negative breast cancer.

In an embodiment, the amount of the an Hsp90 inhibitor administered is from about 2 mg/m² to about 500 mg/m², for example, from about 100 mg/m² to about 500 mg/m², from about 125 mg/m² to about 500 mg/m², from about 150 mg/m² to about 500 mg/m² or from about 175 mg/m² to about 500 mg/m². In an embodiment, the amount of the an Hsp90 inhibitor administered is about 100 mg/m² to about 300 mg/m², from about 125 mg/m² to about 300 mg/m², from about 150 mg/m² to about 300 mg/m² or from about 175 mg/m² to about 300 mg/m². In some embodiments, the amount of the an Hsp90 inhibitor administered is about 2 mg/m², 4 mg/m², about 7 mg/m², about 10 mg/m², about 14 mg/m², about 19 mg/m², about 23 mg/m², about 25 mg/m², about 33 mg/m², about 35 mg/m², about 40 mg/m², about 48 mg/m², about 49 mg/m ², about 50 mg/m², about 65 mg/m², about 75 mg/m², about 86 mg/m², about 100 mg/m², about 110 mg/m², about 114 mg/m², about 120 mg/m², about 144 mg/m², about 150 mg/m², about 173 mg/m², about 180 mg/m², about 200 mg/m², about 216 mg/m² or about 259 mg/m².

The language “twice-weekly” includes administration of a an Hsp90 inhibitor two times in about 7 days. For example, the first dose of the an Hsp90 inhibitor is administered on day 1, and the second dose of the an Hsp90 inhibitor may be administered on day 2, day 3, day 4, day 5, day 6 or day 7. In some embodiments, the twice-weekly administration occurs on days 1 and 3 or days 1 and 4.

In some embodiments, the Hsp90 inhibitor is cyclically administered twice-weekly. For example, the Hsp90 inhibitor is administered for a first period of time, followed by a “dose-free” period, then administered for a second period of time. The language “dose-free” includes the period of time in between the first dosing period and the second dosing period in which no an Hsp90 inhibitor is administered to the subject. A preferred cycle is administering the an Hsp90 inhibitor at a dose described above two times during the week for three consecutive weeks followed by one dose-free week. This cycle is then repeated, as described below.

The language “one cycle” includes the first period of time during which the an Hsp90 inhibitor is administered, followed by a dose-free period of time. The dosing cycle can be repeated and one of skill in the art will be able to determine the appropriate length of time for such a cyclical dosing regimen. In an embodiment, the cycle is repeated at least once. In an embodiment, the cycle is repeated two or more times. In an embodiment, the cycle is repeated 3, 4, 5, 6, 7, 8, 9, 10, 11, 12, 13, 14, 15 or more times, or as many times as medically necessary as determined by one of skill in the art, e.g., as long as the subject exhibits a response with no dose limiting toxicities. In an embodiment, the cycle is repeated until the patient has been determined to be in partial remission (e.g., 50% or greater reduction in the measurable parameters of tumor growth) or complete remission (e.g., absence of cancer). One of skill in the art can determine a patient's remission status using routine methods well known in the art.

The language “pharmaceutically acceptable salt” includes salts prepared from a an Hsp90 inhibitor by reacting the phenolic functional groups and a pharmaceutically acceptable inorganic or organic base. Suitable bases include hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or trialkylamines; dicyclohexylamine; tributyl amine; pyridine; N-methyl,N-ethylamine; diethylamine; triethylamine; mono-, bis-, or tris-(2-hydroxy-lower alkyl amines), such as mono-, bis-, or tris-(2-hydroxyethyl)amine, 2-hydroxy-tert-butylamine, or tris-(hydroxymethyl)methylamine, N,N,-di-lower alkyl-N-(hydroxy lower alkyl)-amines, such as N,N-dimethyl-N-(2-hydroxyethyl)amine, or tri-(2-hydroxyethyl)amine; N-methyl-D-glucamine; and amino acids such as arginine, lysine, and the like. A pharmaceutically acceptable salt can also be formed by reacting the amine functional groups and a pharmaceutically acceptable inorganic or organic acid. Suitable acids include hydrogen sulfate, citric acid, acetic acid, oxalic acid, hydrochloric acid (HQ), hydrogen bromide (HBr), hydrogen iodide (HI), nitric acid, hydrogen bisulfide, phosphoric acid, isonicotinic acid, oleic acid, tannic acid, pantothenic acid, saccharic acid, lactic acid, salicylic acid, tartaric acid, bitartratic acid, ascorbic acid, succinic acid, maleic acid, besylic acid, fumaric acid, gluconic acid, glucaronic acid, formic acid, benzoic acid, glutamic acid, methanesulfonic acid, ethanesulfonic acid, benzenesulfonic acid, palmoic acid and p-toluenesulfonic acid.

As used herein, the term “in combination” refers to the use of more than one therapeutic agent (e.g., 2, 3, 4, 5, 6, 7, 8, 9, 10, or more). The use of the term “in combination” does not restrict the order in which the therapeutic agents are administered to a subject afflicted with cancer. A first therapeutic agent, such as a compound described herein, can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent or treatment, such as an anti-cancer agent, to a subject with cancer. In certain embodiments, one agent may be administered more frequently than the other agent such that multiple doses of one agent are administered for each dose of the other agent(s).

In one embodiment, the Hsp90 inhibitor is administered in combination with one or more chemotherapeutic agents.

The chemotherapeutic agent may be administered at the same time as Hsp90, or the chemotherapeutic agent may be administered after Hsp90.

In certain exemplary embodiments, the Hsp90 inhibitor is Genetespib.

The chemotherapeutic agent can selected from, although is not limited to, the taxanes (paclitaxel and docetaxel), fulvestrant, crizotinib, adriamycin (doxorubicin), cisplatin, camptothecin, 5-fluorouracil, analogs thereof, and other chemotherapeutic agents which demonstrate activity against tumours ex vivo and in vivo. In exemplary embodiments, the chemotherapeutic agent is docetaxel.

The an Hsp90 inhibitor and optionally, one or more additional anticancer agents, can be administered to a subject by routes known to one of skill in the art. Examples of routes of administration include, but are not limited to, parenteral, e.g., intravenous, intradermal, subcutaneous, oral, intranasal {e.g., inhalation), transdermal, topical, transmucosal, and rectal administration. The agents can be administered by different routes of administration.

The an Hsp90 inhibitor, and optionally, one or more additional anticancer agents, may be formulated with a pharmaceutically acceptable carrier, diluent, or excipient as a pharmaceutical composition. Pharmaceutical compositions and dosage forms of the invention comprise one or more active ingredients in relative amounts and formulated in such a way that a given pharmaceutical composition or dosage form can be used to treat cancer. Administration in combination does not require co-formulation.

A pharmaceutical composition of the invention is formulated to be compatible with its intended route of administration. In an embodiment, the composition is formulated in accordance with routine procedures as a pharmaceutical composition adapted for intravenous, subcutaneous, intramuscular, oral, intranasal, or topical administration to human beings. In some embodiments, the an Hsp90 inhibitor is formulated at a concentration of 8 mg/mL in 90% v/v PEG 300 and 10% v/v Polysorbate 80 for intravenous administration.

The invention is illustrated by the following examples, which are not intended to be limiting in any way.

### EXAMPLES

### Example 1

### Evaluation of Ganetespib in Combination with Docetaxel Chemotherapy in Non-Small Cell Lung Carcinoma (NSCLC)

Ganetespib is a novel, potent, small molecule inhibitor of Hsp90, a molecular chaperone which is required for the proper folding and activation of many cancer-promoting proteins. Inhibition of Hsp90 by ganetespib leads to the simultaneous degradation of many of these client proteins and the subsequent death or cell cycle arrest of cancer cells dependent on those proteins. A number of Hsp90 client proteins are also involved in the resistance of cancer cells to other anti-cancer treatments, such as chemotherapy. The ability to reduce cancer-cell drug resistance suggests that the combination of ganetespib with chemotherapies or other anti-cancer agents may provide greater benefit than those agents administered alone. In preclinical studies, ganetespib has shown potent anti-cancer activity against a broad range of solid and hematologic cancers, both as a monotherapy and in combination with certain widely used anti-cancer agents.

Ganetespib has been evaluated in a broad range of cancer clinical trials including the GALAXY NSCLC program (GALAXY-1 and GALAXY-2) in combination with docetaxel chemotherapy, and as monotherapy in certain genetically-defined targeted patient populations. A favorable safety profile has been consistently observed across clinical trials, involving over 1,000 patients treated with ganetespib to date. Ganetespib has not shown the serious liver or common ocular toxicities reported with other Hsp90 inhibitors, or the neurotoxicity, bone marrow toxicities, and alopecia characteristic of many chemotherapies. The most common adverse event reported with ganetespib has been transient, mild or moderate diarrhea, which can be prevented or effectively managed with standard supportive care. In the clinical trials conducted to date, ganetespib has shown promising activity both in combination with chemotherapy and as a monotherapy.

Ganetespib in Lung Cancer: GALAXY-1 Phase 2b Trial

The GALAXY-1 trial was initiated in patients with advanced NSCLC who received one prior treatment for advanced disease, i.e., a second-line treatment setting. GALAXY-1 compared treatment with docetaxel (D) alone, which is approved for second-line treatment, vs. treatment with ganetespib (G) plus docetaxel (D). The aims of the study were to:

Evaluate clinical benefit and establish the safety profile of ganetespib in combination with docetaxel relative to docetaxel alone;

Identify the patient populations, by biomarker or other disease characteristics, which may be most responsive to combination treatment; and

Build the clinical and operational experience needed to optimize the design and execution of the GALAXY-2 Phase 3 trial.

The key GALAXY-1 patient populations comprised populations with (1) elevated LDH (eLDH), (2) KRAS mutations (mKRAS) and/or (3) time since diagnosis of cancer of 6 months or greater (chemosensitive). A rationale for selecting a population with elevated LDH is that LDH-A is a marker of HIF-1α activity, and HIF-1α is a Hsp90 client, and drives invasiveness and metastasis. A rationale for selecting a population with KRAS mutation is that RAS signaling kinases are Hsp90 clients, and there is a medical need. A rationale for selecting a population in which the time since diagnosis of cancer is 6 months or greater (chemosensitive population) is that key cell cycle checkpoint/DNA repair kinases are Hsp90 clients. Additionally, key mitochondrial apoptosis pathway proteins are Hsp90 clients.

Patients in both arms of GALAXY-1 received a standard regimen of docetaxel 75 mg/m² on day 1 of a 21-day treatment cycle. Patients in the combination arm also received ganetespib 150 mg/m² on days 1 and 15. Treatment continued until disease progression or until treatment intolerance. To ensure balance of prognostic factors between the two arms, patients were stratified by ECOG performance status (these scales and criteria are used by doctors and researchers to assess how a patient's disease is progressing, assess how the disease affects the daily living abilities of the patient, and determine appropriate treatment and prognosis), baseline lactate dehydrogenase (LDH) level, smoking status, and time since diagnosis of advanced disease.

Rate of disease progression during or following first line chemotherapy is a common stratification factor in salvage-setting (after first-line treatment) lung cancer clinical trials to ensure balance and evaluate any difference in treatment benefit between refractory and chemosensitive patients. Commonly used measures include time since completion of first line chemotherapy, best response to first line therapy, time since initiation of first line therapy, as well as time since diagnosis of advanced disease.

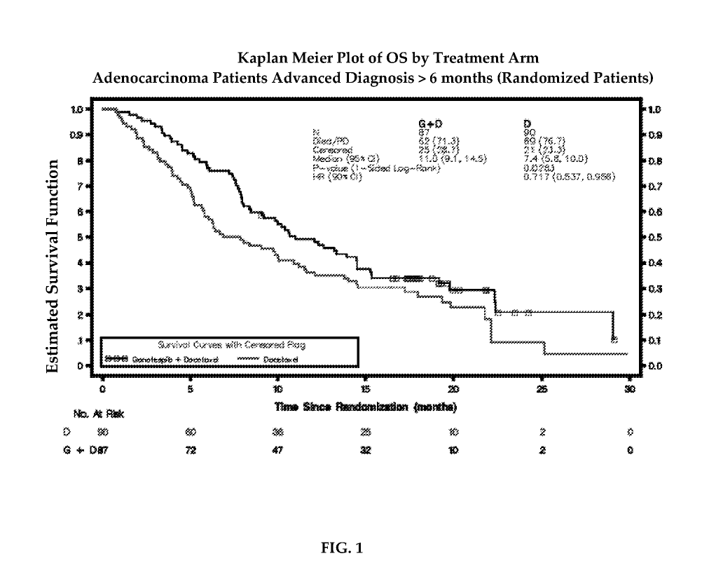

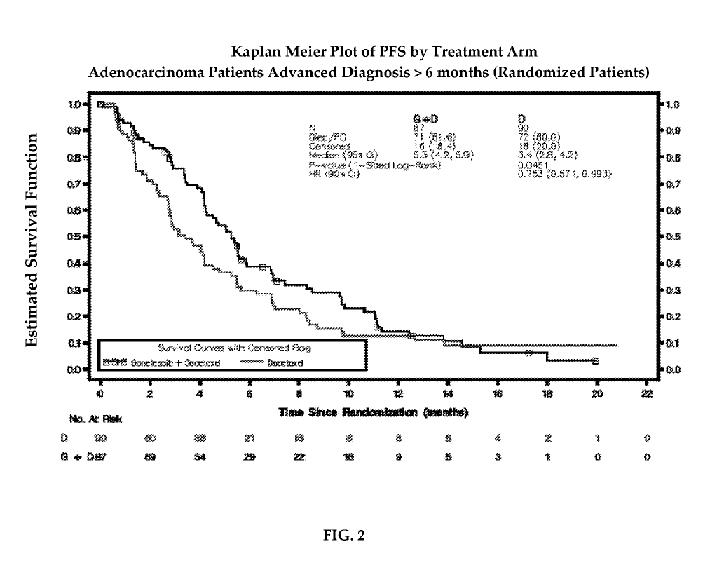

To ensure the specified number of eLDH and mKRAS patients were included, a total of 385 patients were enrolled in GALAXY-1. A summary of key efficacy data from the GALAXY-1 trial is presented in the Table 1 and Table 2, below, and FIGS. 1 and 2.

TABLE 1
Median OS and PFS in Key Patient Populations

| | | | | Chemo- | Adeno- |
|---|---|---|---|---|---|
| | | eLDH | mKRAS | sensitive | carcinoma |
| | G + D vs. D | N = 87 | N = 89 | N = 177 | N = 253 |
| OS | Median | 6.0 vs. 5.1 | 7.6 vs. 6.4 | 11.0 vs 7.4 | 10.2 vs. 8.4 |
| | (months) | | | | |
| | Events | 72 (83%) | 68 (76%) | 132 (75%) | 190 (75%) |
| PFS | Median | 2.8 vs. 2.7 | 3.9 vs. 3.0 | 5.3 vs. 3.4 | 4.5 vs. 3.2 |
| | (months) | | | | |
| | Events | 70 (80%) | 73 (82%) | 142 (80%) | 205 (81%) |

TABLE 2
OS and PFS Hazard Ratios (HRs) in Key Patient Populations

| | Hazard Ratio | | | | |
|---|---|---|---|---|---|
| | G + D vs. D | eLDH | mKRAS | Chemosensitive | Adenocarcinoma |
| | (90% CI) | N = 87 | N = 89 | N = 177 | N = 253 |
| OS | Unadjusted | 0.88 (0.60, 1.30) | 1.18 (0.79, 1.77) | 0.71 (0.53, 0.94) | 0.87 (0.68, 1.10) |
| | | p = 0.300 | p = 0.755 | p = 0.023 | p = 0.150 |
| | Adjusted | 0.75 (0.50, 1.12) | 1.23 (0.81, 1.87) | 0.69 (0.51, 0.93) | 0.84 (0.66, 1.07) |
| | | p-0.118 | p = 0.204 | p = 0.019 | p = 0.114 |
| PFS | Unadjusted | 1.11 (0.75, 1.65) | 0.99 (0.67, 1.46) | 0.75 (0.57, 0.99) | 0.85 (0.67, 1.07) |
| | | p = 0.671 | p = 0.479 | p = 0.045 | p = 0.120 |
| | Adjusted | 0.95 (0.63, 1.42) | 1.18 (0.79, 1.77) | 0.74 (0.56, 0.99) | 0.81 (0.64, 1.03) |
| | | p = 0.409 | p = 0.443 | p = 0.043 | p = 0.073 |

All p-values are 1-sided.

Hazard ratios were calculated with Cox proportional hazards model.
Unadjusted: univariate analysis
Adjusted: pre-specified analysis adjusting for multiple prognostic variables such as gender, smoking status, LDH, ECOG performance status, interval since diagnosis of advanced disease, age, total baseline target lesion size, and geographic region.

FIG. 1 shows an Overall Survival (OS) Kaplan Meier plot for the chemosensitive patient population of GALAXY-1 selected for evaluation in the GALAXY-2 Phase 3 trial. FIG. 2 shows a Progression-Free Survival (PFS) Kaplan Meier plot for the chemosensitive patient population of GALAXY-1 selected for evaluation in the GALAXY-2 Phase 3 trial.

The improvements in OS and PFS were substantially enhanced in patients with diagnosis of advanced disease (e.g. having disease greater than 6 months).

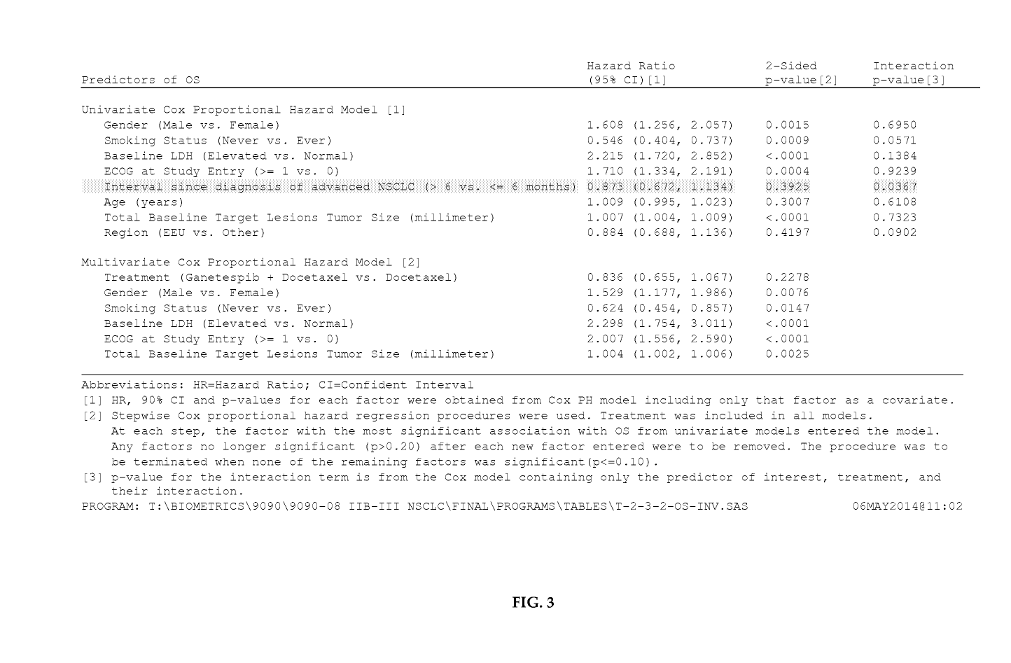

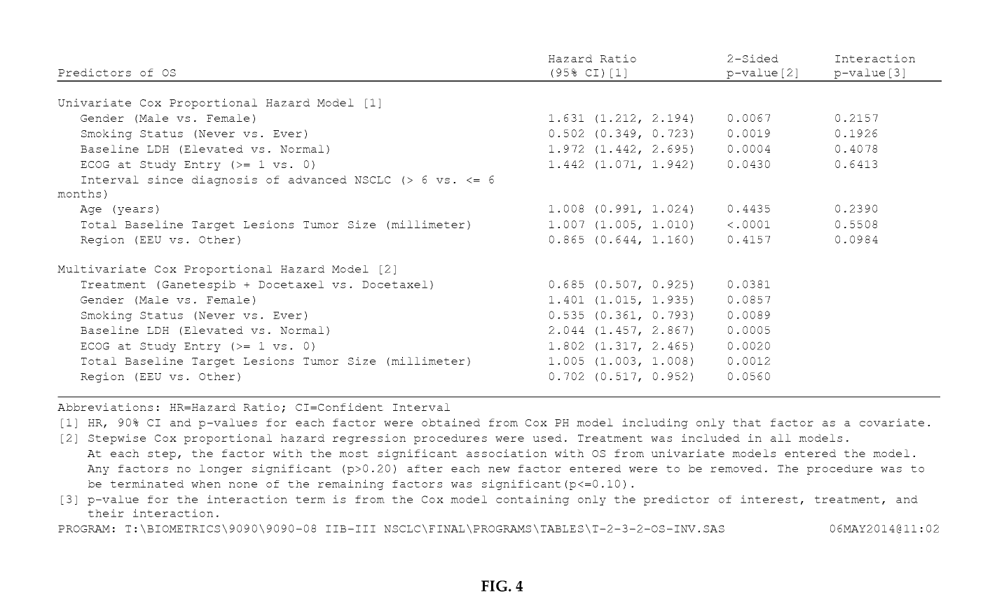

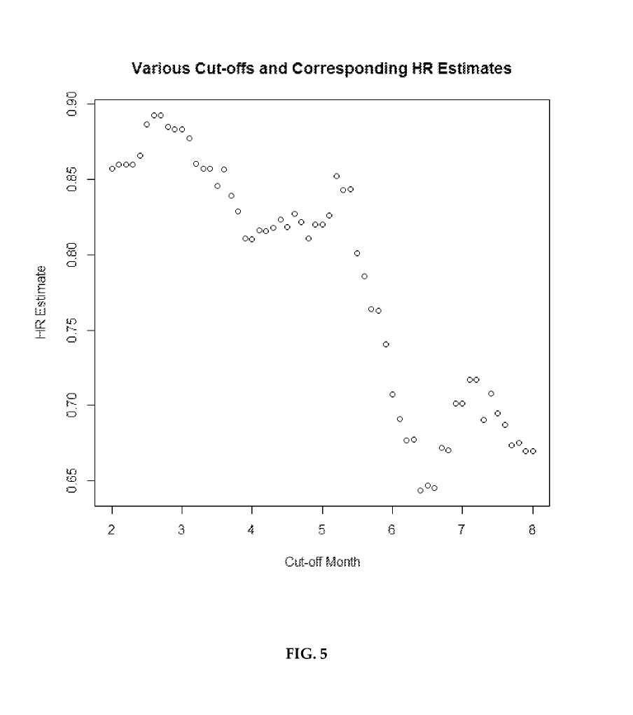

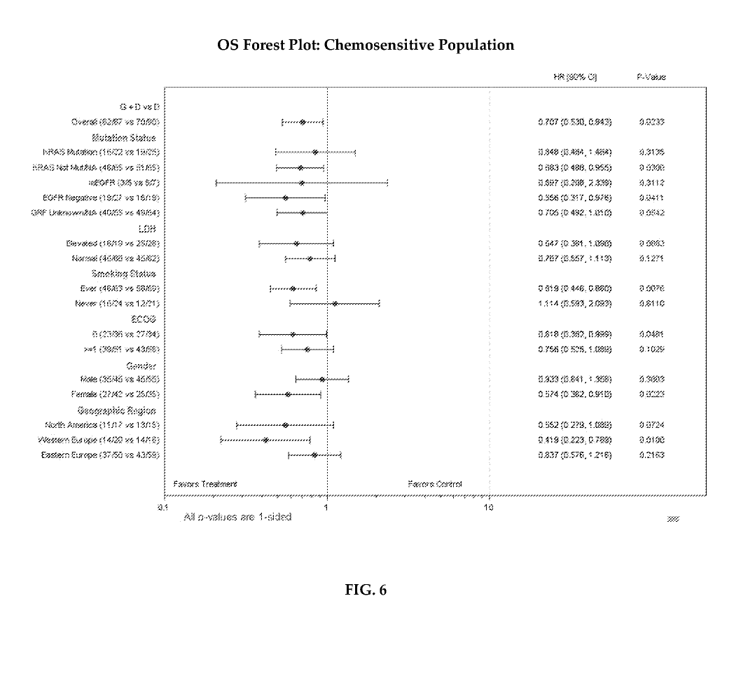

FIGS. 3 and 4 are Tables that show that an interval since diagnosis of advanced non-small cell lunch carcinoma greater than 6 months is a significant prognostic factor on overall survival (p-value<0.05). As shown in FIGS. 3 and 4, an interval since diagnosis of advanced non-small cell lunch carcinoma greater than 6 months is the only significant prognostic factor as compared to gender, smoking status, baseline lactate dehydrogenase (LDH), ECOG at study entry, age, total baseline target lesions tumor size, and region. FIG. 5 is a graph that shows various cut offs (by month) and corresponding hazard ratio (HR) estimates. The hazard ratio is an expression of the hazard or chance of events occurring in the treatment arm as a ratio of the hazard of the events occurring in the control arm. As shown in FIG. 5, at a cut-off of 6 months the HR estimate decreases. FIG. 6 is an overall survival (OS) forest plot for the chemosensitive population.

As shown in the results, ganetespib in combination with docetaxel improved OS and PFS compared to docetaxel alone in the chemosensitive population.

Ganetespib in combination with docetaxel was well tolerated. The safety profile of adenocarcinoma patients treated with the combination of ganetespib (G) and docetaxel (D) was favorable, consistent with previously reported results. The most common adverse events (AEs), all grades, were neutropenia (44% vs. 45%), diarrhea (49% vs. 16%) and fatigue (34% vs. 24%), for G+D (N=123) vs. D (N=126), respectively. Diarrhea was effectively managed with supportive care; the incidence of grade 3 or 4 diarrhea was 4% (G+D) vs. 0% (D). Fatigue was predominantly grade 1 and grade 2; grade 3 or 4 fatigue was 6% (G+D) vs. 4% (D). The most common grade 3 or 4 AEs were neutropenia (38% vs. 42%), febrile neutropenia (9% vs. 4%), and anemia (8% vs. 2%). The proportions of patients with AEs leading to death were 15% vs. 12%, and AEs leading to treatment discontinuation were 7% vs. 6% for G+D vs. D, respectively.

The results of GALAXY-1 validate the choice of the chemosensitive population for the Phase 3 GALAXY-2 study.

GALAXY-2 Phase 3 Trial

The GALAXY-2 trial was a global, randomized, multi-center study comparing the same treatments as in GALAXY-1 in the 2^{nd}-line non-small cell adenocarcinoma patient population, with overall survival as the primary endpoint. Patients were required to be chemosensitive and have tumors that are negative for both EGFR mutation and ALK translocation.

Patients were stratified by ECOG performance status, baseline lactate dehydrogenase (LDH) level, smoking status, and region. Patients on both arms received docetaxel (75 mg/m²) generally for four to six 21-day cycles, according to standard practice at their treatment center. Patients in the combination arm also received ganetespib 150 mg/m2 on days 1 and 15. After completion of docetaxel treatment, patients on the ganetespib arm were eligible to continue to receive ganetespib monotherapy as maintenance treatment.

The GALAXY-2 enrolled approximately 850 patients, of which at least 700 were negative for both ALK translocations and EGFR mutations. Two event-driven interim analyses of the overall survival (OS) primary endpoint of GALAXY-2 were pre-specified. Secondary endpoints included progression free survival (PFS) and Overall survival in elevated LDH populations.

A Phase 1, Pharmacokinetic Study of Ganetespibin Combination with Docetaxel in Subjects with Advanced Solid Tumor Malignancies

This was an open-label, Phase 1 dose-escalation study in patients with solid tumor malignancies. Patients were dosed once-weekly. Dosing was with 150 mg/m² ganetespib (days 1 and 15)+60 mg/m² docetaxel (Day 1). Initial ganetespib dosing regimen was Days 1 and 8; however, Day 8 was later changed to Day 15. Day 4 dosing with single agent ganetespib was added. The dose cycle was 2 weeks+1 week rest.

The primary objective of this study was to determine the recommended doses for the combination of ganetespib and docetaxel in patients with advanced solid tumor malignancies. Secondary objectives were to define the DLTs associated with ganetespib in combination with docetaxel dosing, assess possible PK effects, and evaluate the safety, tolerability, and preliminary evidence of anti-tumor activity of the combination.

A total of 27 patients were enrolled. Of the 27, 20 patients received ganetespib at dose levels of 150 mg/m² or 200 mg/m² in combination with 60-75 mg/m² docetaxel on the Day 1/Day 15 regimen. The remaining 7 patients were on a Day 1, 4, and 15 schedule. (Ganetespib 200 mg/m² with docetaxel 75 mg/m² was the highest dose-combination administered.)

Preliminary data from this Phase 1 study of patients with solid tumors established the recommended Phase 2 dose (150 mg/m² ganetespib in combination with 75 mg/m² docetaxel) and schedule (Days 1 and 15 of each cycle). The study also demonstrated that the combination of ganetespib and docetaxel was well tolerated; the safety profile was similar to docetaxel alone. There was no PK interaction between ganetespib and docetaxel.

A Randomized, Phase 2B/3 Study of Ganetespib in Combination with Docetaxel Versus Docetaxel Alone in Subjects with Stage 3b or 4 Non-Small-Cell Lung Cancer.

This was a randomized, multicenter, parallel group study. Patients in both arms were treated with docetaxel per standard practice until disease progression or intolerability. Patients on both arms were treated until intolerability or disease progression per RECIST.

Key eligibility criteria were Stage IIIB/IV NSCLC; ECOG 0,1; one prior therapy in advanced setting; measurable disease by RECIST; documented disease progression; clinically stable CNS metastases; available tumor tissues; and adequate organ function.

Patients were randomized in a 1:1 ratio to receive either ganetespib in combination with docetaxel or docetaxel alone.

The 2 stages of the study were Stage 1, an open-label, Phase 2b exploratory study that investigates the clinical activity of the combination of ganetespib and docetaxel in NSCLC patients. Data analysis of the Stage 1 data informed choices for the final design and patient population characteristics in Stage 2 (Phase 3 study). Data from patients recruited into Stage 1 was not included in the Stage 2 analysis. Approximately 240 patients were enrolled (120 patients per treatment arm).

Stage 2 was a randomized Phase 3 study designed to evaluate the efficacy of the combination of ganetespib and docetaxel in NSCLC patients. Stage 2 enrolled approximately 500 patients (250 per treatment arm).

The experimental arm was dosed with ganetespib in combination with docetaxel 75 mg/m². Ganetespib (150 mg/m²) and docetaxel (75 mg/m²) were administered on Day 1 of a 3-week cycle. Ganetespib (150 mg/m²) was administered again on Day 15 of each 3-week cycle. Docetaxel 75 mg/m² was administered on Day 1 of a 3-week treatment cycle. 385 patients were enrolled. A total of 381 patients received at least one dose of study drug.

A Randomized, Phase 3 Study of Ganetespib in Combination with Docetaxel Versus Docetaxel Alone in Patients with Advanced Non-Small-Cell Lung Adenocarcinoma

This study was a randomized, multicenter, parallel-group study. Patients were randomized in a 1:1 ratio to receive either ganetespib in combination with docetaxel or docetaxel alone. The study enrolled approximately 500 patients.

Ganetespib was administered in combination with docetaxel. Ganetespib (150 mg/m²) and docetaxel (75 mg/m²) were administered on Day 1 of a 3-week cycle. Ganetespib alone (150 mg/m²) was administered on Day 15 of each 3-week cycle. In the control arm, docetaxel (75 mg/m²) was administered on Day 1 of a 3-week treatment cycle.

This was an open-label, multicenter, randomized Phase 3 study of patients with Stage IIIB/IV NSCLC of adenocarcinoma histology. Eligible patients must have failed only one prior systemic therapy for Stage IIIB/IV disease and have measurable disease as defined by Response Evaluation Criteria in Solid Tumors (RECIST).

The primary objective of this study was to evaluate and compare overall survival (OS) in non-small cell lung cancer (NSCLC) patients with adenocarcinoma histology treated with ganetespib in combination with docetaxel versus docetaxel alone. The study compared efficacy and tolerability in both treatment groups.

Treatment and Ganetespib Maintenance Phase—Docetaxel in either treatment arm was be administered for a maximum number of cycles according to prevailing practice and investigator decision, generally until disease progression, patient's withdrawal of consent, or intolerability. In the combination arm, following completion of docetaxel treatment according to prevailing practice, patients whose disease had not progressed will continued to receive ganetespib alone. This ganetespib maintenance therapy continued until disease progression, patient's withdrawal of consent, or intolerability. Patients will be followed up for survival at approximately 6-week intervals.

All publications cited herein are incorporated by reference in their entirety. In case of conflict, the present specification, including definitions, will control. In addition, the materials, methods, and examples throughout the specification are illustrative only and not intended to be limiting in any way.

1. A method of treating cancer in a subject, comprising administering an effective amount of an Hsp90 inhibitor to the subject, wherein the time since diagnosis of cancer in the subject is 6 months or greater.

2. The method of claim 1, comprising the step of selecting a subject in which the time since diagnosis of cancer in the subject is 6 months or greater.

3. The method of claim 1, wherein the cancer is a solid tumor or a hematological malignancy.

4. The method of claim 1, wherein the cancer is advanced cancer.

5. The method of claim 1, wherein the cancer is selected from the group consisting of: primary cancer, metastatic cancer, breast cancer, colon cancer, rectal cancer, lung cancer, oropharyngeal cancer, hypopharyngeal cancer, esophageal cancer, stomach cancer, pancreatic cancer, liver cancer, gallbladder cancer, bile duct cancer, small intestine cancer, urinary tract cancer, kidney cancer, bladder cancer, urothelium cancer, female genital tract cancer, cervical cancer, uterine cancer, ovarian cancer, choriocarcinoma, gestational trophoblastic disease, male genital tract cancer, prostate cancer, seminal vesicle cancer, testicular cancer, germ cell tumors, endocrine gland tumors, thyroid cancer, adrenal cancer, pituitary gland cancer, skin cancer, hemangiomas, melanomas, sarcomas arising from bone and soft tissues, Kaposi's sarcoma, brain cancer, nerve cancer, ocular cancer, meningial cancer, astrocytoma, glioma, glioblastoma, retinoblastoma, neuroma, neuroblastoma, Schwannoma, meningioma, solid tumors arising from hematopoietic malignancies, leukemia, Hodgkin's lymphoma, non-Hodgkin's lymphoma, Burkitt's lymphoma, metastatic melanoma, recurrent or persistent ovarian epithelial cancer, fallopian tube cancer, primary peritoneal cancer, epithelial ovarian cancer, primary peritoneal serous cancer, non-small cell lung cancer, gastrointestinal stromal tumors, colorectal cancer, small cell lung cancer (SCLC), melanoma, glioblastoma multiforme, non-squamous non-small-cell lung cancer, malignant glioma, primary peritoneal serous cancer, metastatic liver cancer, neuroendocrine carcinoma, refractory malignancy, triple negative breast cancer, HER2 amplified breast cancer, squamous cell carcinoma, nasopharageal cancer, oral cancer, biliary tract, hepatocellular carcinoma, squamous cell carcinomas of the head and neck (SCCHN), non-medullary thyroid carcinoma, neurofibromatosis type 1, CNS cancer, liposarcoma, leiomyosarcoma, salivary gland cancer, mucosal melanoma, acral/lentiginous melanoma, paraganglioma; pheochromocytoma, advanced metastatic cancer, solid tumor, squamous cell carcinoma, sarcoma, melanoma, endometrial cancer, head and neck cancer, rhabdomysarcoma, multiple myeloma, gastrointestinal stromal tumor, mantle cell lymphoma, gliosarcoma, bone sarcoma, and refractory malignancy.

6. The method of claim 5, wherein the cancer is non-small cell lung carcinoma (NSCLC).

7. The method of claim 1, wherein the Hsp90 inhibitor is administered in combination with one or more chemotherapeutic agents.

8. The method of claim 7, wherein the chemotherapeutic agent is administered at the same time as Hsp90.

9. The method of claim 7, wherein the chemotherapeutic agent is administered after Hsp90.

10. The method of claim 1, wherein the Hsp90 inhibitor is Genetespib.

11. The method of claim 7, wherein the chemotherapeutic agent is selected from the group consisting of: the taxanes (paclitaxel and docetaxel), fulvestrant, crizotinib, adriamycin (doxorubicin), cisplatin, camptothecin, 5-fluorouracil, analogs thereof, and other chemotherapeutic agents which demonstrate activity against tumours ex vivo and in vivo.

12. The method of claim 11, wherein the chemotherapeutic agent is docetaxel.

13. The method of any one of claims 1-12, wherein the cancer was previously treated and not responsive.

14. (canceled)

15. The method of claim 1, wherein the amount of the Hsp90 inhibitor administered is from 75 mg/m² to 260 mg/m².

16. The method of claim 1, wherein the amount of the Hsp90 inhibitor administered is from 125 mg/m² to 260 mg/m².

17. The method of claim 1, wherein the amount of the Hsp90 inhibitor administered is from 175 mg/m² to 260 mg/m².

18. The method of claim 1, wherein the amount of the Hsp90 inhibitor administered is 75 mg/m², 85 mg/m², 100 mg/m², 110 mg/m², 115 mg/m², 120 mg/m², 145 mg/m², 150 mg/m², 175 mg/m², 180 mg/m², 200 mg/m², 215 mg/m² or 260 mg/m².

19. The method of claim 7, wherein the Hsp90 inhibitor is administered by intravenous infusion.

20. (canceled)

21. (canceled)

22. A method of treating advanced NSCLC in a subject, comprising:
selecting a subject in which the time since diagnosis of cancer in the subject is 6 months or greater; and
administering Genetespib to the subject in combination with docetaxel, thereby treating advanced NSCLC in the subject.

23. A kit to practice the method of claim 1.
